(19) United States
(12) Patent Application Publication
(10) Pub. No.: US 2006/0258994 A1
(43) Pub. Date: Nov. 16, 2006

(54) Implantable delivery device for administering pharmacological agents to an internal portion of a body

(76) Inventor: Robert L. Avery, Santa Barbara, CA (US)

Correspondence Address: Daniel J. Meaney, Jr, P.O. Box 22307, Santa Barbara, CA, 93121, US

(21) Appl. No.: 11/415,054

(22) Filed: May 1, 2006

Related U.S. Application Data
(60) Provisional application No. 60/680,320, filed on May 12, 2005.

(51) Int. Cl. A61M 35/00

(52) U.S. Cl. 604/294

(57) ABSTRACT

An implantable refillable device to deliver pharmacologic agents through a sclera to an internal portion of an eye is shown. The device comprises a hollow reservoir and a delivery tube. The delivery tube has a proximal end and a distal end. The proximal end communicates with the hollow reservoir and the distal end terminates in a dispensing outlet. The delivery tube is configured in a selected shape to extend from the hollow reservoir anterior in an eye to a posterior segment posterior in an eye adjoining a sclera. The second end is configured to position the dispensing outlet in contact with or contiguous the sclera and is located posteriorly in an eye and proximate to an internal portion of an eye to be treated with a pharmacologic agent. An implantable device to deliver pharmacologic agents through an outer surface tissue of an organ is also shown.

### CROSS-REFERENCES TO RELATED APPLICATIONS

This Application claims the benefit, under Title 35, United States Code §119(e), of U.S. Provisional Patent Application Ser. No. 60/680,320 filed May 12, 2005.

### STATEMENT REGARDING FEDERALLY SPONSORED RESEARCH OR DEVELOPMENT

Not Applicable

### REFERENCE TO A “MICROFICHE APPENDIX” (SEE 37 CFR 1.96)

Not Applicable

### BACKGROUND OF THE INVENTION

1. Field of the Invention

The present invention relates generally to an implantable device to deliver pharmacological agents or drugs to an internal portion of an eye and more particularly to an implantable refillable device to deliver pharmacological agents directly through a sclera to an internal portion of an eye. In the preferred embodiment, the internal portion of the eye is the macula. In an alternate embodiment, a therapeutic agent or drug is delivered to the optic nerve. This invention also relates to a method of using the device to treat an eye. In one embodiment, an implantable device to deliver pharmacologic agents through an outer surface tissue of an organ comprises a hollow reservoir and a delivery tube having a dispensing outlet wherein the delivery tube is configured in a selected shape to extend from the hollow reservoir to a selected portion of the outer surface tissue of an organ.

2. Description of the Prior Art

It is known in the art to implant a medical device in the eye to facilitate delivery of pharmacological agents or drugs, into the eye.

U.S. Pat. Nos. 5,725,493; 5,830,173 and 6,251,090 disclose an intravitreal medicine delivery device that is in the form of an implantable device for delivering drugs and other pharmacological agents into the vitreous cavity of an eye. An incision is required through the eye wall (sclera) at the pars plana and into the vitreous cavity to enable an intravitreal extension from the implantable device to project into the vitreous cavity and administer the pharmacological agents.

U.S. Pat. No. 5,836,935 discloses an implantable refillable controlled release device to deliver drugs directly to an internal portion of the body. The device includes a hollow reservoir and a drug delivery tube communicating with the hollow reservoir. The drug delivery tube includes at least one rate-limiting permeable membrane that regulates drug delivery. The preferred embodiment of the device is adapted to deliver a drug to a brain tumor at a controlled rate.

U.S. Pat. No. 5,824,072 discloses a biocompatible ocular implant comprising active agents which are employed for introduction into a suprachoroidal space or avascular region of an eye for therapeutic purposes. The implant, which is refillable, is surgically located adjacent to the vitreous and the pars plana between optic part of the retina and lens.

U.S. Pat. No. 6,852,106 discloses an implantable, refillable rate controlled drug delivery device that includes a base structure having at least one opening and a second opening and the base structure defines a chamber. A septum covers the first opening and is configured to substantially prevent leakage from the first opening to an exterior surface of the device. A drug delivery tube comprising a first and second distal end and the first distal end communicated with the chamber through the second opening. At least one rate-limiting permeable membrane is disposed across a passageway between the base structure and the second distal of the drug delivery device. The membrane passively regulates drug delivery..

U.S. Pat. No. 5,904,144 discloses a capsule which is surgically placed in a desired location of an eye. The capsule includes cells which produce a biologically active molecule. The capsule also includes a surrounding biocompatible jacket through which the biologically active molecule may diffuse into the eye. The biocompatible jacket may immunoisolate the encapsulated cells protecting the same from attack by the immune system of the patient.

U.S. Pat. No. 6,713,081 discloses an ocular implant for delivery of a therapeutic agent to an eye in a controlled manner. The ocular implant administers a therapeutic agent in either a single mode or a dual mode release kinetics. In one embodiment, the ocular implant delivers drugs continuously to the eye by initial delivery at a high release rate to eye tissue soon after placement of the implant in or near the eye, followed by a continuous, sustained lower release rate thereafter.

U.S. Pat. No. 6,375,972 discloses sustained release drug delivery device for a mammalian organism. The device includes an inner core or reservoir including the effective agent, an impermeable tube that encloses portions of the reservoir and a permeable member at the end of the tube.

U.S. Pat. Nos. 6,669,950 and 6,416,777 disclose a drug delivery device for a human eye. The drug delivery device includes a pharmaceutically active agent within the body of the device and a geometry that facilitates the implantation of the device on an outer surface of the sclera and beneath the inferior oblique muscle. The pharmaceutically active agent is disposed above the macula. When the body no longer contains the pharmaceutically active agent, the device can be surgically be removed and replaced with a new device containing the pharmaceutically active agent.

United States Published Patent Application Publication Number US 2003/0064088 discloses a surgically implantable and sealable delivery device having interface window or port which exposes therapeutic agent with an organ or tissue to be treated. The sealable delivery device may be affixed to the organ or tissue by use of an adhesive to provide protective diffusion of a therapeutic agent into the tissue to avoid leakage to surrounding tissue.

As is well known to those skilled in the art, several diseases and conditions of the posterior segment of the eye diminish and destroy eyesight. The leading causes of legal blindness in the developed world are diabetic retinopathy and macular degeneration. Several of the drug delivery devices described in the above United States Patents can be used to deliver pharmacologic agents to provide treatment of such eye diseases.

As a result, there is an increased interest in the development and administration of pharmacologic agents to treat these diseases, as well as other diseases involving the posterior segment of the eye, such as glaucoma and optic neuropathies.

One known prevalent disease that is the leading cause of blindness in older patients is wet age related macular degeneration commonly known as wet AMD. Wet AMD is caused by an overgrowth of newly formed choroidal blood vessels [choroidal neovascularization (CVN)] in the macula. The macula is part of the retina and enables fine detailed vision.

When the blood vessels leak blood or fluid, such leakage causes the macula to swell which causes distorted vision and decreased central vision. In many cases, the peripheral visions remains functional. Wet AMD affects an estimated 1.6 million United States patients, is growing at the rate of about 200,000 United States patients per year and results in approximately forty percent (40%) of the United States population over the age of 75 and approximately twenty percent (20%) of the United States Population over the age of 60 suffering from some degree of macular degeneration.

Known treatments for wet AMD include thermal lasers and drug injections. Thermal lasers are directed into the eye cauterizing the new blood vessels and may halt or slow the progression of the disease. The thermal laser treatment leaves a permanent blind spot where the AMD disease has occurred.

There is one known as photodynamic therapy which comprises an intravenous injection of a drug, Visudyne, followed by a low powered laser treatment to the retina. The Visudyne drug becomes chemically activated when exposed to a cold laser. The treatment results in inhibiting the blood vessel leakage and slows visual loss. As a result of the wet AMD disease, new treatments and drugs are being developed which require effective medical devices for delivering drugs to the posterior section of the eye. As a result, several experimental drugs are being used to treat wet AMD in order to maintain sight or reduce the rate at which a patient is losing sight.

Such experimental drugs include Rataane from Alcon, Inc. and Macugen from Eyetech Pharmaceuticals. Retaane is in the form of a steroid that inhibits the development of new blood vessels in the macula. Retaane is delivered adjacent the eye via a canula system having a narrow tube which is positioned around the outer surface of the sclera, the fibrous tissue that covers most of the eyeball. The drug is delivered towards the back or posterior segment of the eye directly in contact with the external sclera without actually puncturing the eye.

Macugen is injected directly into the vitreous cavity of the eyeball of a patient.

Many pharmacologic agents administrated as eye drops usually do not penetrate to the posterior part of the eye at therapeutic levels. Systematic administration of pharmacologic agents can also be limited by difficulty of the pharmacologic agents to penetrate the blood-ocular barriers and by the potential systemic side effects of these agents.

As a result of the above, certain of the known drug delivery devices, such as the devices disclosed in U.S. Pat. Nos. 5,725,493; 5,830,173 and 6,251,090 discussed above, have been developed to deliver and administer pharmacologic agents including drugs directly to the vitreous cavity to treat the affected portions of the eye.

Other drug delivery devices, such as the devices disclosed in U.S. Pat. Nos. 6,669,950 and 6,416,777 discussed above, have been developed to deliver and administer pharmacologic agents or drugs directly to sclera overlying the affected portions of an eye located in the posterior segments of the eye. Some pharmacologic agents, such as anacortate acetate, have been found to penetrate through the sclera sufficiently to avoid the need to inject anacortate acetate directly into the vitreous cavity.

In delivering anacortate acetate adjacent to the eye using a canula system having a narrow tube which is positioned around the outer surface of the sclera, leakage from the incision sight resulting in a certain portion of the pharmacologic agent not being effectively delivered to the back or posterior segment of the eye, an effect generally referred to as “reflux”. As such, the efficacy of the pharmacologic agent was serious impeded as a result of the “reflux” of the canula system used as a drug delivery device. When “reflux” of a pharmacologic agent occurs following a simple juxtascleral injection or treatment, the efficacy of the pharmacologic agent including drugs has been shown to be significantly less.

Thus, the known drug delivery devices have the above described limitations and problems. There is, therefore, need for improvements in drug delivery devices which can efficiently, effectively and economically deliver repeated injections of pharmacologic agents or drugs to the sclera while avoiding “reflux” at the injection site.

### BRIEF SUMMARY OF THE INVENTION

The present invention discloses a new, novel and unique pharmacologic agent or drug delivery device which provides for repeated injections of pharmacologic agent or drug delivery to the sclera while avoiding “reflux” at the injection site thereby increasing the efficacy of the administered pharmacologic agent or drugs.

The present invention discloses and teaches a new, novel and unique pharmacologic agents or drug deliver device in the form of an implantable, refillable device to deliver pharmacologic agents, or a drug, through the sclera to an internal portion of an eye.

In the preferred embodiment, the device includes a hollow reservoir and a delivery tube having a first end or proximal end and a second end or distal end. The proximal end communicates with the hollow reservoir and the distal end terminates in a dispensing outlet. The delivery tube is configured in a shape, e.g. generally arcuate shape, curved shape or a bent shape, to extend from the hollow reservoir anterior in an eye to a posterior segment posterior in an eye adjoining a sclera. The distal end is configured to position the dispensing outlet in contact with or contiguous the outer surface of a sclera posteriorly in an eye and proximate to an internal portion of an eye to be treated with the pharmacologic agent. The pharmacologic agent may be in the form a drug to treat a specific disease of the eye, e.g. wet AMD. When the pharmacologic agent is injected into the hollow reservoir, the delivery tube transports the pharmacologic agent through the dispensing opening and directly into that portion of the sclera in contact with or contiguous the dispensing outlet whereupon the pharmacologic agent penetrates the sclera and passes therethrough to the internal portion of the eye to be treated.

Accordingly, one advantage of the present invention is that the implantable refillable device can be used for administrating repeated and doses of pharmacological agents to the posterior segment of the eye to treat an internal portion of an eye using a simple in office injection.

Another advantage of the present invention is that the implantable refillable device is configured to eliminate reflux during administration of a pharmacologic agent thereby increasing the efficacy of the administration of the pharmacologic agent to the sclera at the posterior segment of an eye and subsequent treatment of an internal portion of an eye.

Another advantage of the present invention is that the implantable refillable device has a delivery tube wherein the dispensing outlet defined by the distal end of the delivery tube may be configured in the form of an unobstructed opening to enable a pharmacologic agent to pass therethrough.

Another advantage of the present invention is that the implantable refillable device has a delivery tube wherein the dispensing outlet defined by the distal end of the delivery tube may be configured in the form of a semi-permeable or permeable membrane which is selected to have a porosity to pass a pharmacologic agent therethrough.

Another advantage of the present invention is that the implantable refillable device has a delivery tube wherein the dispensing outlet defined by the distal end of the delivery tube may be configured in the form of a permeable membrane or semi-permeable to allow a controlled rate of delivery of the pharmacologic agent.

Another advantage of the present invention is that the implantable refillable device drug has a delivery tube wherein the dispensing outlet defined by the distal end of the delivery tube may be configured in the form of a permeable membrane which is selected to have a porosity to pass a pharmacologic agent therethrough and to have sufficient structural integrity to resist tissue from the outer surface of a sclera migrating in to and occluding the pharmacologic agent dispensing opening.

Another advantage of the present invention is that the implantable refillable device geometry enables the distal end of the delivery tube end to have a dispensing outlet to overlie the sclera above or proximate the macula.

Another advantage of the present invention is that the implantable refillable device geometry is configured to hug or stay close to or adjoin the scleral surface and curved inferior to the inferior oblique muscle and be positioned to overlie the macula region of the sclera.

Another advantage of the present invention is that the implantable refillable device has a delivery tube having a distal end that defines a dispensing outlet for providing contact with or contiguous a large area of the sclera including the portions of the sclera located over the macula and beneath two rectus muscle enabling the dispensing outlet to administer a pharmacologic agent over a large surface area of the sclera.

Another advantage of the present invention is that the implantable refillable device hollow reservoir may have a self-sealing needle injection section or injection port.

Another advantage of the present invention is that the implantable refillable device hollow reservoir may have a self-sealing needle injection section or injection port and an impermeable shield spaced distally from the needle injection section or injection port to prevent further penetration of a needle of a syringe after entering the needle injection section or injection port.

Another advantage of the present invention is that the implantable refillable device delivery tube may be configured to include a self-sealing elastic flap at the distal end thereof to allow for egress of excess pharmacologic agent into the orbital space if the hollow reservoir is over filled via the needle injection section or injection port.

Another advantage of the present invention is that the implantable refillable device delivery tube may be configured to have the second end or distal end defining a dispensing outlet positioned about or surrounding the optic nerve proximate the area where the optic nerve inserts into the sclera.

Another advantage of the present invention is that the implantable refillable device drug delivery tube may be configured to have the second end or distal end define a dispensing outlet in the form of a semi-circular member and of a dimension to enable the semi-circular member to partially encircle the optic nerve as the optic nerve inserts into the sclera.

Another advantage of the present invention is that the implantable refillable device delivery tube may be configured to have the second end or distal end defining a dispensing outlet formed to couple with a optic nerve sheath fenestration adjacent to the second end.

Another advantage of the present invention is that the implantable refillable device delivery tube may be configured to have the second end or distal end defining a dispensing outlet formed as a canula which extends into an optic nerve sheath fenestration adjacent to the second end to enable administration of pharmacological agents directly through the optic nerve sheath fenestration to treat the optic nerve.

Another advantage of the present invention is that the implantable refillable device delivery tube may be configured to have the second end or distal end defining a dispensing outlet formed as a canula which extends into an optic nerve sheath fenestration adjacent to the second end to enable administration of pharmacological agents directly through the optic nerve sheath fenestration to treat the optic nerve and the dispensing outlet defined by the distal end may be configured in the form of a semi or permeable membrane to allow a controlled rate of delivery of the pharmacologic agent.

Another advantage of the present invention is that the implantable refillable device may be configured to include an adhesive strip of a medical grade adhesive to facilitate adhesion of the device to the scleral surface.

Another advantage of the present invention is that the implantable refillable device may be configured at the proximal end, which is the anterior end of the device, with suture eyelets to attach or fix the device securely to the sclera to allow penetration of a needle of a syringe through the needle injection section or injection port with moving the device.

Another advantage of the present invention is that the implantable refillable device may be used in a method for treating an internal portion in a posterior segment of an eye through a sclera with a pharmacological agent.

Another advantage of the present invention is that the implantable refillable device may be treated or impregnated with pharmacologic agents to minimize fibrosis and scaring around the device.

Another advantage of the present invention is that the implantable refillable device may be used in a method for treating an internal portion in a posterior segment of an eye through a sclera with repeated doses or injections of a pharmacological agent in a simple office based procedure.

Another advantage of the present invention is that the implantable refillable device may be refilled to allow repeated administration of a pharmacological agent or, if filled with a pharmacological agent, that pharmacologic agent can be removed by replacement of the pharmacologic agent with sterile air and the sterile air can then be exchanged with another pharmacological agent.

Another advantage of the present invention is that the implantable refillable device may be used in a method for treating an internal portion of an eye using a pharmacological agent.

Another advantage of the present invention is that an implantable dispensing device can be implanted in a tunnel formed in the sclera wherein the sclera tissue surrounds and seals the dispensing outlet of the dispensing device within the tunnel.

Another advantage of the present invention is that the implantable dispensing device can be removablely affixed to an insertion tool to facilitate insertion of the implantable dispensing device within a tunnel formed in the sclera and upon implantation the insertion tool can be removed enabling the implantable device to be sealed in position by the sclera enabling the pharmacological agent to be administered through the tissue of the sclera wherein significantly increases the efficacy of the administration of the pharmacological agent to the sclera.

Another advantage of the present invention is that the implantable device using the teaching of this invention can be used to deliver pharmacologic agents through an outer surface tissue of an organ. The implantable device may comprise a hollow reservoir and a delivery tube having a dispensing outlet wherein the delivery tube is configured in a selected shape to extend from the hollow reservoir to a selected portion of the outer surface tissue of an organ. The dispensing outlet can be configured to be positioned in contact with or contiguous an outer tissue of an organ and proximate to an internal portion of a body to be treated with a pharmacologic agent.

Another advantage of the present invention is that the implantable device can be configured as an implantable delivery device for delivering a therapeutic agent into a target tissue. The implantable delivery device may comprise an enclosed therapeutic agent container having a dispensing outlet and the container may be configured to be implanted in a tunnel or flap of the outer tissue of an organ wherein the dispensing outlet is engaged by and sealed within the tunnel by the outer tissue of an organ enabling administration of a therapeutic agent into the target tissue contiguous to the dispensing outlet.

### BRIEF DESCRIPTION OF THE DRAWING

The present invention will become more fully understood from the following detailed description of a preferred but non-limiting embodiment thereof, described in connection with the accompanying drawings, wherein:

FIG. 1 is a pictorial representation of one embodiment of an implantable refillable device for delivering pharmacological agents according to the present invention wherein the device is implanted on a human eyeball and the pictorial representation illustrates an eyelid being lifted to expose the region of the eye where the implant is located;

FIG. 2 is a pictorial representation similar to FIG. 1 illustrating the needle injection section or injection port being located at the proximal end of the implantable refillable device and that the dispensing tube extends posteriorly from the device;

FIG. 3 is a three dimensional schematic representation of the human eye showing one embodiment of an implantable refillable device for delivering pharmacological agents according to the present invention as shown in FIG. 1 wherein the interior oblique muscle passes over the implant and the distal end of the delivery tube is positioned on the sclera and located over the macula;

FIG. 4 is a schematic representation of the anterior view of a human eye showing the proximal end of another embodiment of an implantable refillable device having a delivery tube configured to be inserted under the oblique muscle and which defines a dispensing outlet in the form of large opening to be positioned over the macula and in contact with or contiguous the sclera and wherein the device is located on the temporal segment of the eye and has self-sealing needle injection section or injection port located at the anterior section of the device;

FIG. 5 is a schematic representation of the posterior view of a human eye of FIG. 4 showing the distal end of the delivery tube defining a dispensing outlet in the form of large opening to be positioned over the macula and in contact with or contiguous with the sclera;

FIG. 6 is a pictorial representation of another embodiment of an implantable refillable device for delivering pharmacological agents according to the present invention which is implanted on a human eyeball wherein the dispensing tube distal end defines a dispensing outlet having a permeable membrane;

FIG. 7 is a cross-section taken along section line 7-7 of FIG. 6 showing that the distal end of the dispensing tube is positioned proximate the optic nerve and that the dispensing outlet has a permeable membrane;

FIG. 8 is a schematic representation of the anterior view of a human eye showing the proximal end of yet another embodiment of an implantable refillable device having a delivery tube configured to define a dispensing outlet at the distal end thereof in the form of large opening having a large surface area to be positioned in the superior temporal quadrant to increase the surface area in contact with or contiguous the sclera and wherein the device has self-sealing needle injection section or injection port located at the anterior section of the device;

FIG. 9 is a schematic representation of the posterior view of a human eye of FIG. 8 showing the dispensing outlet in the form of large winged opening having a large surface area positioned in the superior temporal quadrant to increase the surface area in contact with or contiguous the sclera;

FIG. 10 is a schematic representation of the anterior view of a human eye showing the proximal end of still yet another embodiment of an implantable refillable device wherein the distal end of the delivery tube is configured to define a dispensing outlet in the form of a semi-circular member and the device has an self-sealing needle injection section or injection port located on the temporal segment of the eye;

FIG. 11 is a schematic representation of the posterior view of a human eye of FIG. 10 showing the distal end of the delivery tube of the another embodiment of the an implantable refillable device wherein the distal end of the delivery tube is configured to define a dispensing outlet in the form of a semi-circular member and of a dimension to enable the semi-circular member to partially encircle or surround the optic nerve as the optic nerve inserts into the sclera;

FIG. 12 is a front, left side and top perspective view of the embodiment of the implantable refillable device shown in FIG. 1;

FIG. 13 is a cross-sectional view of the implantable refillable device shown in FIG. 1 having a dispensing opening configured in the form of an unobstructed opening to enable a pharmacologic agent to pass therethrough;

FIG. 14 is a front plan view of the implantable refillable device shown in FIG. 1 having a self-sealing needle injection section or injection port located at the anterior section of the device and including suture eyelets to attach or fix the device securely to the sclera to allow penetration of a needle of a syringe through the self-sealing needle injection section or injection port without moving the device;

FIG. 15 is a cross-sectional view of another embodiment of the implantable refillable device having a annular shape or curved shape to conform to the curvature of the eyeball and wherein the dispensing outlet is configured in the form of an unobstructed opening to enable a pharmacologic agent to pass therethrough and the alternate use of a permeable membrane that can be located across the unobstructed opening being shown by dashed lines;

FIG. 16 is a pictorial representation of another embodiment of a implantable refillable device having a discernable reservoir at the anterior section of the device wherein the reservoir has a self-sealing needle injection section or injection port, a dispensing tube in communication with the hollow reservoir wherein the dispensing tube is shaped to bend around the inferior oblique muscle during implanting and the second end or distal end is configured in the form of an dispensing opening to engage the sclera;

FIG. 17 is a cross-sectional view of another embodiment of the implantable refillable device having a distal end configured in the form of a semi-circular member and of a dimension to enable the semi-circular member to partially encircle or surround the optic nerve as the optic nerve inserts into the sclera and wherein the dispensing opening is configured in the form of an unobstructed opening to enable a pharmacologic agent to pass there through;

FIG. 18 is a cross-sectional view of another embodiment of the implantable refillable device having a distal end configured to in the form of a semi-circular member and of a dimension to enable the semi-circular member to partially encircle or surround the optic nerve as the optic nerve inserts into the sclera and which is similar to the device of FIG. 17 and wherein the dispensing outlet is configured in the form of a permeable membrane in the outlet to enable a pharmacologic agent to pass therethrough;

FIG. 19 is a cross-sectional view of another embodiment of the implantable refillable device having a distal end configured to define a dispensing outlet at the distal end thereof in the form of large opening having a large surface area to be positioned in the superior temporal quadrant to increase the surface area in contact with or contiguous the sclera and wherein the wherein the dispensing outlet is configured in the form of an unobstructed opening to enable a pharmacologic agent to pass therethrough;

FIG. 20 is a cross-sectional view of another embodiment of the implantable refillable device having a distal end configured to define a dispensing outlet at the distal end thereof in the form of large opening having a large surface area to be positioned in the superior temporal quadrant to increase the surface area in contact with or contiguous the sclera similar to the device of FIG. 19 and wherein the dispensing outlet is configured in the form of a permeable membrane in the outlet to enable a pharmacologic agent to pass therethrough;

FIG. 21 is a cross-sectional view of still yet another embodiment of an implantable refillable device similar to the device shown in FIG. 1 having an impermeable shield in the form of spaced baffles spaced distally from the needle insertion section or injection port to prevent further penetration of a needle of a syringe after entering the needle insertion section or injection port;

FIG. 22 is a cross-sectional view of still yet another embodiment of an implantable refillable device similar to the device shown in FIG. 1 having an impermeable shield in the form of a serpentine delivery tube having compound curves defining a serpentine pathway wherein the compound curves and spaced distally from the needle insertion section or injection port to prevent further penetration of a needle of a syringe after entering the needle insertion section or injection port;

FIG. 23 is a pictorial representation of the layers of the eye between the sclera and vitreous cavity;

FIG. 24 is a pictorial representation of a tunnel formed in the sclera as shown by dashed lines to accommodate a implantable dispensing device wherein the dispensing opening is implanted and surrounded by the sclera;

FIG. 25A is a pictorial representation of the sclera having a tunnel formed therein as shown by dashed lines to accommodate the implantation of a single use dispensing device within the sclera and showing an insertion tool having the implantable dispensing device having a pharmacological agent contained within the housing;

FIG. 25B is a pictorial representation of the sclera having a tunnel formed therein as shown by dashed line in FIG. 25A wherein the dispensing device illustrated in 25A which is removablely attached to an insertion tool has been positioned at the desired location to implant the implantable dispensing device within the tunnel in the sclera;

FIG. 25C is a pictorial representation of the sclera having a tunnel formed therein as shown by dashed line in FIG. 25B wherein the dispensing device illustrated in 25B has been implanted at the desired location in the sclera within the tunnel wherein the sclera tissue surrounds and seals the dispensing device within the tunnel and the insertion tool has been removed; and

FIG. 26 is a pictorial representation of an embodiment of the insertable implantable dispensing device filled with a slow release pharmacologic agent enabling the device to administer through the delivery tube to administer the pharmacological agent through the dispensing outlet into the tissue of the sclera.

### DETAILED DESCRIPTION OF THE INVENTION

Before proceeding with a detailed description of the invention, it would be helpful for a better understanding of the invention and in order to appreciate the significance, uniqueness and novelty of the teachings of the invention to provide a review of the background of the eye including the elements thereof including the internal portions of the posterior segment of the eye that can be treated using the teachings of the present invention.

### Background

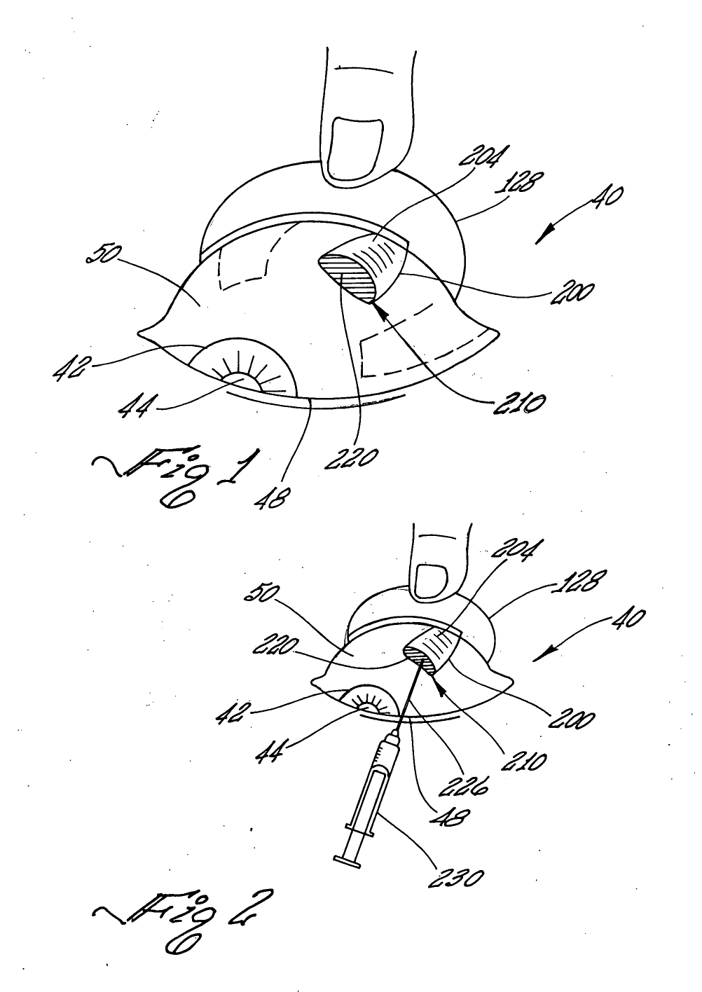

The preferred embodiments of the present invention and their advantages are best understood by referring to FIGS. 1 through 26 of the drawings, like numerals being used for like and corresponding parts of the various drawings.

FIGS. 1 through 8 illustrate various elements of the human eye important to an understanding of the present invention and the relationship thereof relative to the various internal portions of the eye to be treated using the teachings of this invention. Referring first to FIGS. 1 through 8, elements of a human eye, shown generally by arrow 40, are schematically illustrated. Eye 40 has a cornea 42, a lens 44, vitreous 48, a sclera 50, a choroid 52, a retina 56, and an optic nerve 60. Eye 40 is generally divided into an anterior segment 64 and a posterior segment 68. Anterior segment 64 of eye 40 generally includes the portions of eye 40 anterior of ora serata 70. Posterior segment 68 of eye 40 generally includes the portions of eye 40 posterior of ora serata 70. Retina 56 is physically attached to choroid 52 in a circumferential manner proximate pars plana 104, posteriorly to optic disk 106. Retina 56 has a macula 110 located slightly lateral to optic disk 106. As is well known in the ophthalmic art, macula 110 is comprised primarily of retinal cones and is the region of maximum visual acuity in retina 56. A Tenon's capsule or Tenon's membrane 120 (a breakway portion be illustrated in FIG. 7) is disposed on sclera 50. As can be seen from FIG. 3, the eye 40 is within its orbit 140. The inferior oblique muscle 142 runs under lateral rectus muscle 144. The insertion line 142′ of inferior oblique muscle 142 into sclera 50 is located just above the superior border of lateral rectus muscle 144. The position of the inferior oblique muscle 142 in a right human eye 40 is a mirror image to its position on left human eye. Cornea 42, conjunctiva 124, superior rectus muscle 150, inferior rectus muscle 152, superior oblique muscle 158 and limbus 126 are also shown in FIG. 3.

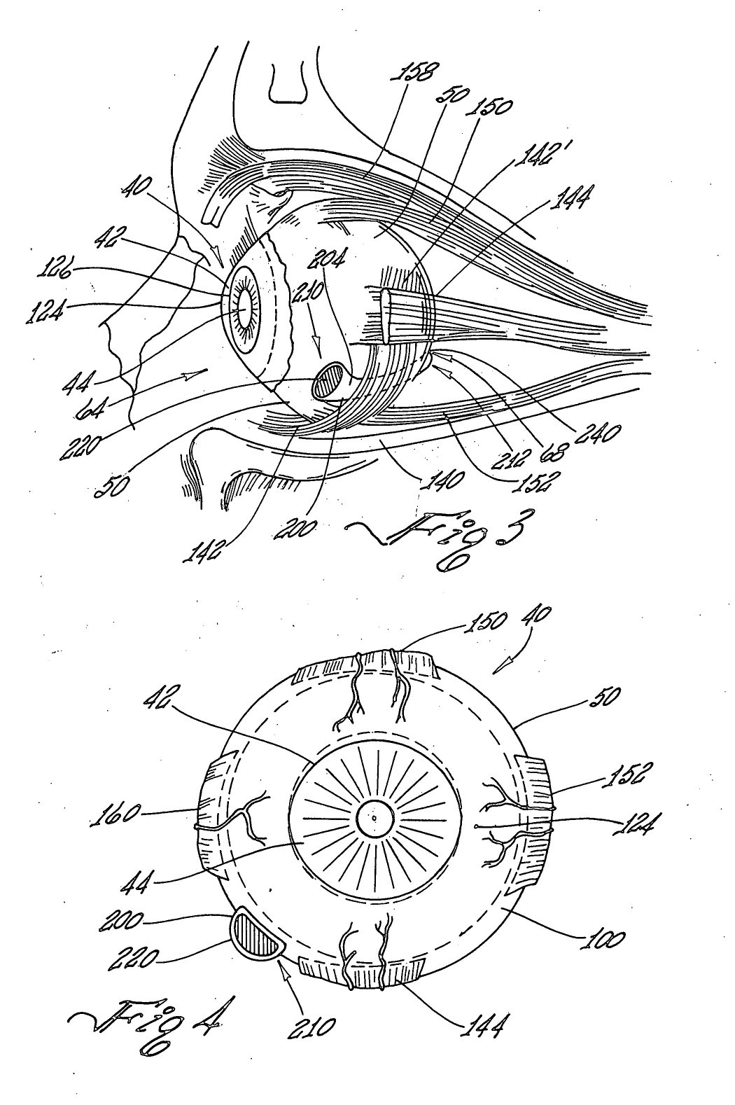

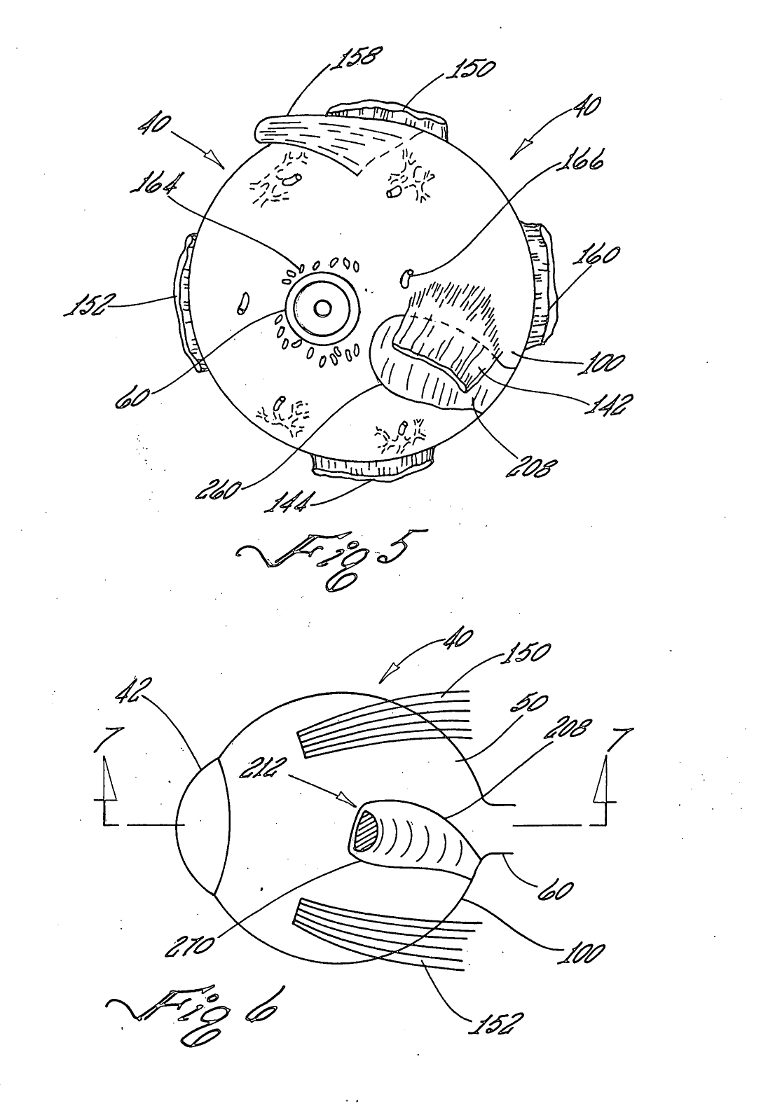

FIGS. 4 and 8 schematically illustrate an anterior view of the eye 40 with its four recti muscles, the superior rectus muscle 150, the medial rectus muscle 160, the inferior rectus muscle 152 and the lateral rectus muscle 144.

FIG. 5, a posterior view of the eye 40, also illustrates the four recti muscles 150, 160, 152 and 144 and shows the cillary vessels 164 and the long cilliary arteries 166.

### Description of Implantable Refillable Device

The pictorial representation of in FIGS. 1 and 2 are of one embodiment of an implantable refillable device, shown generally by 200, for delivering pharmacological agents or drugs to a posterior segment 68 shown in FIG. 7 to treat an internal portion of an eye 40 located below or proximate the sclera 50. The device 200 is implanted on the human eyeball 40. The eyelid 128 is depicted as being lifted to expose the region of the eye on the sclera 50 where the device 200 is located.

The implantable refillable device 200 delivers pharmacologic agents through a sclera 50 to an internal portion of an eye such as the macula 110 or the optic nerve 60, both being shown in FIG. 7.

The device 200 includes a hollow reservoir 204 having an internal cavity, shown in greater detail in FIG. 13, for receiving the pharmacological agent to be used for treating an internal portion of the eye.

The hollow reservoir 204 has a first end or proximal end shown by arrow 210 and a second end or distal end shown by arrow 212 as shown in FIG. 13.

The proximal end 210 of the hollow reservoir 204 is the anterior section of the device 200. The proximal end 210 has a self-sealing needle insertion section 220 to enable a needle 226 of a syringe 230 to be passed through the needle insertion section 220 and into the internal cavity of the hollow reservoir 204 to enable filling of the internal cavity with a pharmacologic agent to be dispensed in an internal portion of an eye 40. As shown in FIGS. 1 and 2, the dispensing tube 208, shown in FIG. 13, extends posteriorly from the device 200.

The three dimensional schematic representation of the human eye of FIG. 3 shows the embodiment of an implantable refillable device 200 for delivering pharmacological agents according to the present invention as shown in FIG. 1. The interior oblique muscle 142 passes over the device 200 and the distal end 212 of the delivery tube 208 is positioned on the sclera 50 and located over the macula 110, shown in FIG. 7.

The delivery tube 208 has a first end 210 and a second end 212. The first end 210 communicates with the hollow reservoir 204. The second end 212 terminates in a dispensing outlet 240. The delivery tube 208 is configured in a selected shape, generally and generally arcuate shape or curved shape, to extend from the hollow reservoir 204, anterior in an eye, to a posterior segment 68 posterior in an eye adjoining a sclera 50. However, the shape of the tube may include a deflecting portion to enable the device during insertion to bend around the inferior oblique muscle as discussed in connection with FIG. 16 hereinbelow.

The second end 212 is configured to position the dispensing outlet 240 contiguous an outer surface portion of a sclera 50 located posteriorly in an eye and proximate to an internal portion of an eye to be treated with a pharmacologic agent.

The schematic representation of the anterior view of a human eye 40 of FIG. 4 and the posterior view of a human eye 40 of FIG. 5 show another embodiment of an implantable refillable device 200. In FIG. 4, the proximal end 210 has the self-sealing needle insertion section 220 for receiving a needle for injection of a pharmacological agent into the hollow reservoir 204.

In FIG. 5, the proximal end 210 of the device 200 has a delivery tube 208 configured to be inserted under the inferior oblique muscle 142. The delivery tube 208 defines at the distal end 212 thereof a dispensing outlet having a selected surface area in the form of large opening 260 to be positioned on the sclera 50 proximate or over the macula and in contact with or contiguous the sclera 50. The device 200 may be in The device 200 is located on the temporal segment of the eye 40 and has self-sealing needle injection section or needle injection port 220 located at the anterior section, the proximal end 210, of the device 200 as shown in FIG. 5.

The pictorial representation in FIG. 6 is of another embodiment of an implantable refillable device 200 for delivering pharmacological agents according to the present invention. The implantable refillable device 200 is shown implanted on a human eyeball 40 and the dispensing tube 208 distal end 212 defines a dispensing outlet 240 having a semi-permeable membrane or permeable membrane 270.

The cross-section taken along section line 7-7 of FIG. 6 is shown in FIG. 7 and shows in detail the macula 110 and optical disk 106 of the optic nerve 60 which are internal portions of the eye 40. As shown in FIG. 6, the distal end 212 of the dispensing tube 208 is positioned proximate the optic nerve 60 and over the macula 110 and the dispensing outlet 240 has a permeable membrane 270.

The schematic representation of the anterior view of a human eye 40 of FIG. 8 and the posterior view of a human eye 40 of FIG. 9 show yet another embodiment of an implantable refillable device 200 having a delivery tube 208 configured to define a dispensing outlet 240 at the distal end 212 thereof in the form of large opening.

FIG. 8 shows the proximal end 210 of the device 200, which is the anterior portion of the device 200, having the self-sealing needle insertion section or injection port 220 at the first end or proximal end 210 of the device 200. The device 200 is located between the medial rectus muscle 160 and the superior rectus muscle 150.

The implantable refillable device 200 of FIG. 9 has the delivery tube 208 configured to define a dispensing outlet 240 at the distal end 212 thereof in the form of large opening shown by arrow 280 which has a large surface area and is positioned in the superior temporal quadrant of the sclera 50 so as to increase the surface area in contact with or contiguous the sclera 50. The device 200 is implanted on the temporal segment of the eye 40 and has self-sealing needle injection section or injection port 220 located at the anterior section of the device 200.

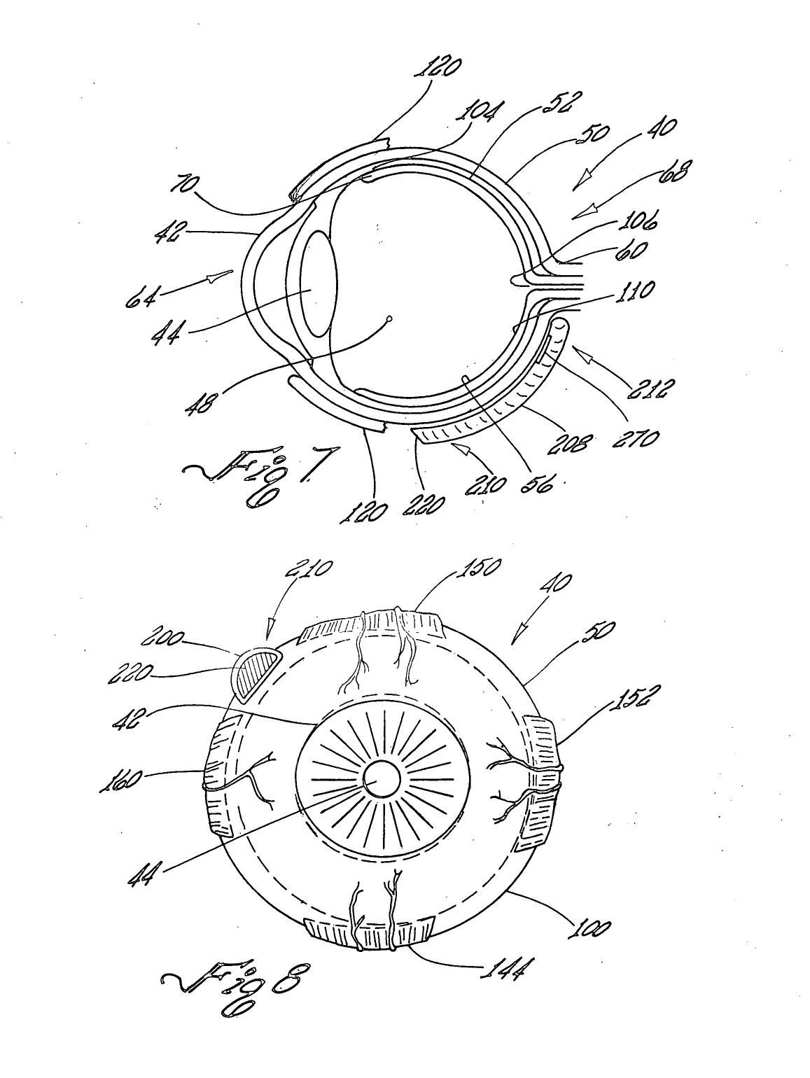

The schematic representation of the anterior view of a human eye of FIG. 10 and the posterior view of a human eye 40 of FIG. 11 show still yet another embodiment of an implantable refillable device 200 wherein the distal end 212 of the delivery tube 208 is configured to define a dispensing outlet 240 in the form of a semi-circular member shown by arrow 290.

In FIG. 10, shows the proximal end 210 of the device 200, which is the anterior section of the device 200, having the self-sealing needle insertion section or injection port 220 at the first end or proximal end 210 of the device 200. The device 200 is located between the superior rectus muscle 150 and the inferior rectus muscle 152.

FIG. 11 shows that the distal end 212 of the delivery tube 208 is configured to define a dispensing outlet 240 in the form of a semi-circular member shown by arrow 290. The dispensing outlet 240 at the distal end 212 of the delivery tube 208 is in the form of a semi-circular member 290 and is of a dimension to enable the semi-circular member 290 to partially encircle or surround the optic nerve 260 as the optic nerve inserts into the sclera 50.

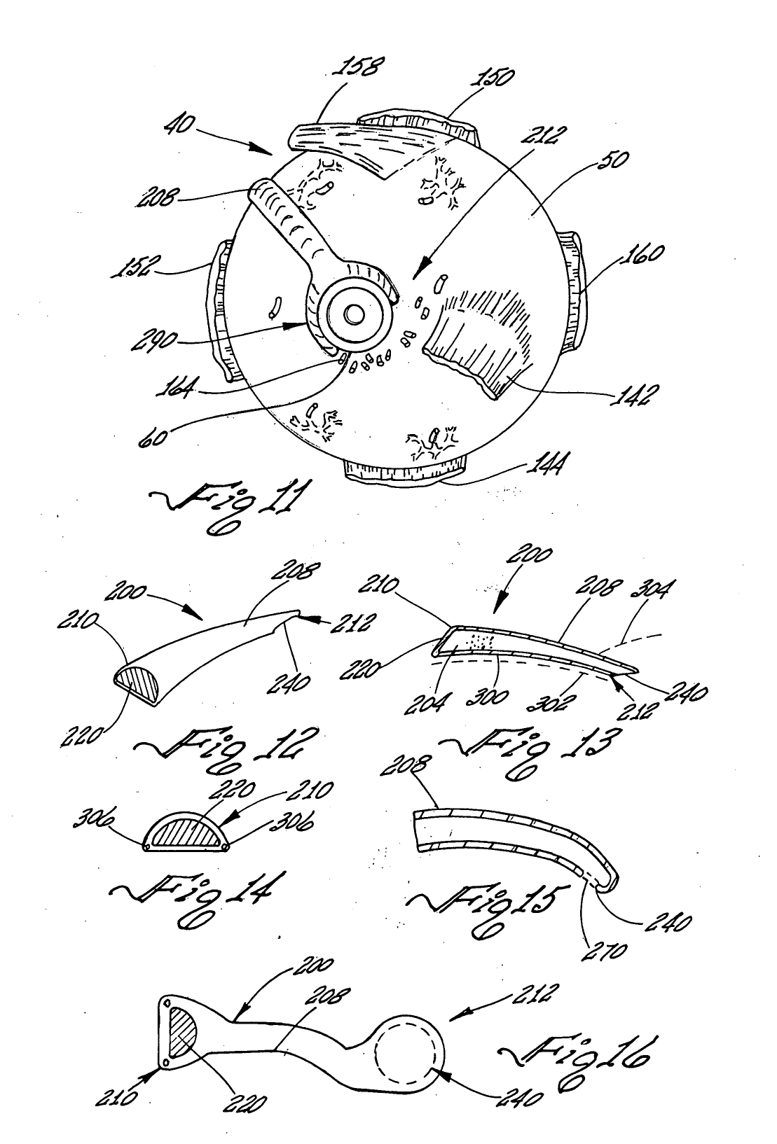

As discussed below, FIGS. 17 and 18 show that the semi-circular member 290 can be in the form of an unobstructed opening or in an opening having a permeable membrane.

In FIGS. 12, 13 and 14 are of the embodiment of the implantable refillable device 200 shown in FIG. 1.

The implantable refillable device 200 is configured to deliver pharmacologic agents or drugs through a posterior outer surface of an eye, namely the sclera, to an internal portion of an eye. As shown in FIGS. 12, 13 and 14, the device 200 includes a hollow reservoir 204 having an internal cavity.. The anterior section of the device 200 includes a needle insertion section or injection port 220. The needle insertion section 220 is configured to retain a pharmacological agent or drug located within the internal cavity of the hollow reservoir 204 from refluxing over the eye 40. The needle insertion section 220 is positioned relative to the anterior portion of an eye 40 to enable a needle 226 of a syringe 230, shown in FIG. 2, to be passed through the needle insertion section 220 and into the internal cavity 240 to enable filling of the internal cavity with a pharmacological agent or drug and for withdrawing of a pharmacological agent or a drug from the internal cavity.

The delivery tube 208 has a proximal end 210 and a distal end 212. The proximal end 210 communicates with the hollow reservoir 204 and the distal end 212 terminates in a dispensing outlet 240. The delivery tube 208 is configured in a shape, generally curved shape or annular shape, to extend from the hollow reservoir 204, anterior in an eye 40, to a posterior segment 68 posterior in an eye 40 and adjoining the sclera 50.

Depending on the structure of the device 200, the device 200 may adjoin the sclera, that is may touch the sclera at some point, or be contiguous the sclera wherein the device 200 is in contact with the sclera most of the time. Also, it is envisioned that an adhesive stripe of a medical grade adhesive may be affixed to the device 200 to facilitate adhesion of the device to the scleral surface. The term “adjoining” the sclera is meant to cover all such implantation conditions.

The distal end 212 is configured to position the dispensing outlet 240 in contact with or contiguous the posterior surface of the sclera 50in an eye 40.

In FIG. 13, the cross-sectional view of the implantable refillable device 200 shows a dispensing opening 240 configured in the form of an unobstructed opening to enable a pharmacologic agent to pass therethrough. The needle injection section 220 enables the injecting into the hollow reservoir 204 of a pharmacologic agent or a controlled released drug, shown by dashed spheres 300 in FIG. 13, by a needle 226, e.g. a needle of a syringe as shown in FIG. 2, to be used for treating an eye. The controlled released drug may be configured in the form of micro-spheres having a known dissolution rate for providing a controlled rate of drug delivery through the dispensing outlet for treating an eye.

As shown in FIG. 13. the distal end 212 may be configured to include a self-sealing elastic flap shown as dashed line 304 to allow for egress of excess pharmacologic agent into the orbital space if the hollow reservoir 204 is over filled via the needle injection section or injection port 220.

The device 200 illustrated in FIG. 14 is may be configured to have suture eyelets 306 formed therein to attach or fix the device securely to the sclera to allow penetration of a needle of a syringe through the needle injection section or injection port 220 without moving the device.

In the alternative, the device 200 may include an adhesive stripe of a medical grade adhesive affixed thereto as depicted by dashed line 308 in FIG. 13 to facilitate adhesion of the device 200 to the surface of the sclera 50.

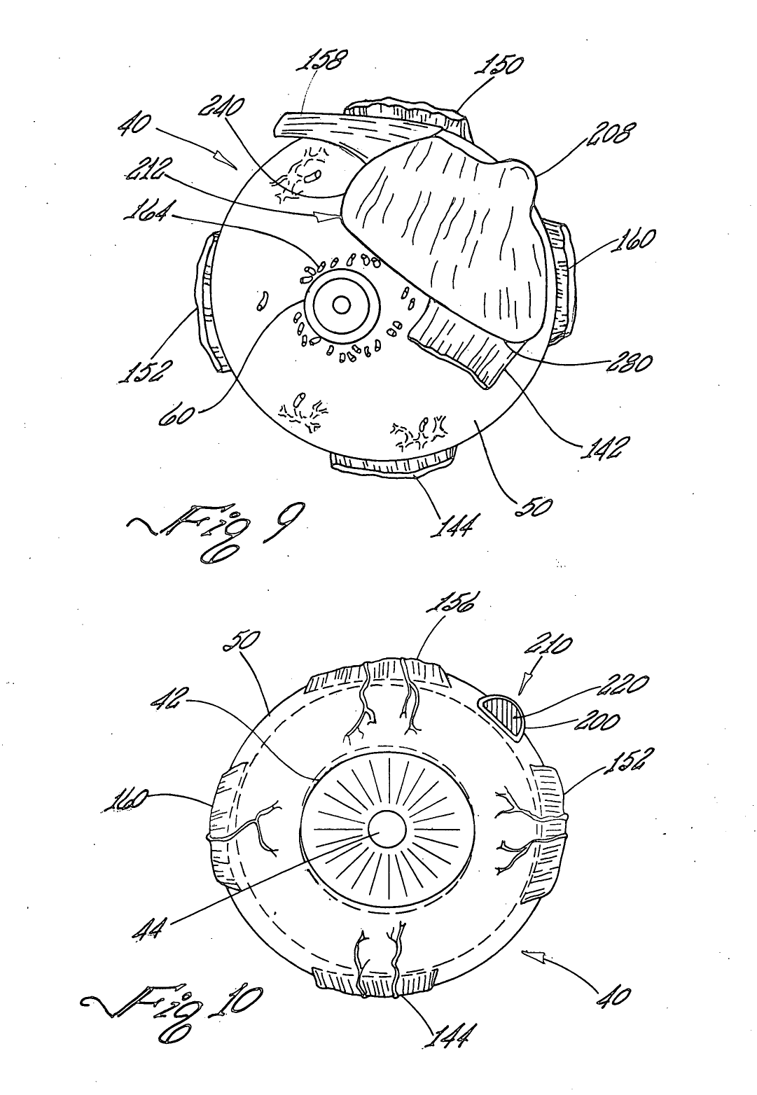

FIG. 15 is a cross-sectional view of another embodiment of the implantable refillable device 200 in a curved shape or annular shape to conform to the curvature of the eyeball 40. The distal end 212 of dispensing tube 208 is configured to position the dispensing outlet 240 on the sclera 50. The dispensing outlet 240 is configured in the form of an unobstructed opening to enable a pharmacologic agent or drug to pass therethrough. The alternate use of a semi-permeable membrane or permeable membrane that can be located across the unobstructed opening is shown by dashed lines 270.

FIG. 16 is a pictorial representation of another embodiment of a implantable refillable device 200 having a discernable hollow reservoir 204 at the anterior section of the device 220, the proximal end 210 of the dispensing tube 208 wherein the hollow reservoir 204 has a self-sealing needle injection section or injection port 220, a dispensing tube 208 in communication with the hollow reservoir 204 wherein the dispensing tube 208 is shaped to bend around the inferior oblique muscle 142 during implanting and the second end or distal end 212 of the dispensing outlet 240 is configured in the form of an circular shaped dispensing opening to engage the sclera 50.

FIG. 17 is a cross-sectional view of another embodiment of the implantable refillable device 200 having a distal end 212 configured in the form of a semi-circular member 290. The semi-circular member 290 may appear in the shape or form of a wrench and is of a dimension to enable the semi-circular member 290 to partially encircle the optic nerve 60 as the optic nerve 60 inserts into the sclera 50 as shown in FIG. 11. The dispensing outlet 240 is in the form of an opening which is configured in the form of an unobstructed opening to enable a pharmacologic agent to pass therethrough.

FIG. 18 is a cross-sectional view of another embodiment of the implantable refillable device 200 similar to the device 200 of FIG. 17 but wherein the dispensing outlet in the form of the semi-circular member 290 is configured in the form of a permeable membrane 270 located in the dispensing outlet 240 to enable a pharmacologic agent to pass therethrough.

FIG. 19 is the cross-sectional view of the other embodiment of the implantable refillable device of FIGS. 19 and 20. The device 200 has a distal end that is configured to define a dispensing outlet 240 at the distal end 212 thereof in the form of large opening 320 having a large surface area to be positioned in the superior temporal quadrant of the sclera 50 to increase the surface area in contact with or contiguous the sclera 50. The dispensing outlet 240 is configured in the form of an unobstructed opening to enable a pharmacologic agent to pass therethrough.

A shown in the device 200 of FIG. 20, which is similar to the device 200 of FIG. 19, the dispensing outlet 240 at the distal end 212 thereof is in the form of large opening 320 having a large surface area to be positioned in the superior temporal quadrant of the sclera 50, the large opening 320 may have a permeable membrane 270 formed thereacross in the outlet to enable a pharmacologic agent to pass therethrough.

FIG. 21 is a cross-sectional view of still yet another embodiment of an implantable refillable device 200 similar to the device shown in FIG. 1 wherein the device has an impermeable shield 330 located in the hollow reservoir 204 and positioned therefrom in the form of spaced baffles 332 spaced distally from the needle injection section or injection port 220 to prevent further penetration of a needle of a syringe after entering the needle injection section 220.

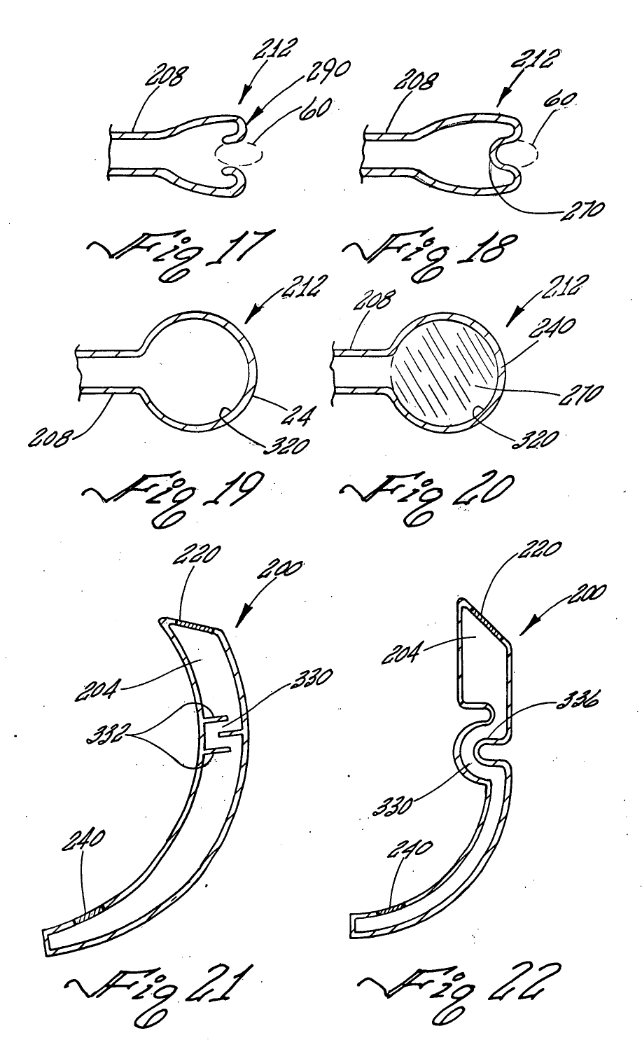

FIG. 22 is a cross-sectional view of still yet another embodiment of an implantable refillable device 200 similar to the device shown in FIG. 1 and 21 having an impermeable shield 330 in the form of a serpentine delivery tube having compound curves 336 defining a serpentine pathway wherein the compound curves 336 are spaced distally from the needle injection section or injection port 220 to prevent further penetration of a needle of a syringe after entering the needle injection section 220.

It is envisioned that the implantable refillable device of the present invention can have the distal end of the dispensing tube fabricated with an opening to cooperate with an in an optic nerve sheath fenestration.

### Method of Using Implant

A method for treating an eye using the implantable refillable device of the present invention is envisioned as part of the teachings of the present invention. The method may comprise the steps of: (a) forming an implantable refillable device to deliver a pharmacologic agent to an internal portion of an eye wherein the device has a hollow reservoir and a delivery tube having a first end and a second end wherein the first end communicates with the hollow reservoir, wherein the delivery tube is configured in a shape to extend from the hollow reservoir anterior in an eye to a posterior segment posterior in an eye adjoining a sclera and wherein the second end terminates in a dispensing outlet to be position in contact with or contiguous a sclera of an eye; and (b) surgically implanting the device under the Tenon's capsule and in contact with or contiguous the sclera of an eye with the hollow reservoir positioned anteriorly on the sclera at a location where a needle can be inserted into the hollow reservoir and with the dispensing outlet being located posteriorly on the sclera and proximate the location of an internal portion of an eye to be treated by a pharmacological agent.

The method for treating an eye wherein the step of surgically implanting further includes positioning the dispensing outlet onto the sclera proximate the macula of an eye.

The method for treating a wherein the step of surgically implanting further includes positioning said dispensing outlet onto the sclera proximate the optic nerve of an eye.

The method for treating a wherein the step of surgically implanting further includes implanting includes positioning said dispensing outlet onto a sclera proximate the optic nerve of an eye.

The method for treating an eye wherein the step of surgically implanting further includes implanting positioning said dispensing outlet onto a sclera surrounding the optic nerve of an eye.

The method for treating an eye may further comprise the step of injecting through a needle into the hollow reservoir a pharmacologic agent to be used for treating an eye.

The method for treating an eye may further comprise the step of injecting through a needle into the hollow reservoir a controlled released drug to be used for treating an eye.

The method for treating an eye may further comprise the step of injecting through a needle into the hollow reservoir a controlled released drug configured in the form of micro-spheres having a known dissolution rate for providing a controlled rate of drug delivery through the dispensing outlet for treating an eye.

The method for treating an eye may further comprise the step of injecting through a needle into the hollow reservoir a pharmacologic agent comprising anacortate acetate which penetrates through the sclera.

The method for treating an eye may further comprise the step of withdrawing through a needle from the hollow reservoir a pharmacologic agent by the steps of alternating injecting sterile air through the needle injection section and withdrawing the pharmacologic agent through the needle.

The self-sealing needle injection section of the implantable refillable device is configured to have one or more needles inserted sequentially into and removed from the self-sealing needle insertion section. This enables the self-sealing needle insertion section to be used for administrating repeated juxtasclera injections into the posterior segment of the eye to treat an internal portion of an eye or for removal of a pharmacological agent from the hollow reservoir in situ.

In order to remove a pharmacologic agent from the hollow reservoir, the following process may be used. The method is performed by placing the eye in a dependent location, inserting a needle through the needle insertion section, injecting a small amount of sterile air through the needle into the hollow reservoir, allowing bubbles of sterile air to rise to the superior part of the hollow reservoir, withdrawing an appropriate quantity of pharmacologic agent from the hollow cavity using the needle, alternating injecting another small amount of sterile air through the needle into the hollow reservoir and withdrawing through the needle an appropriate quantity of pharmacologic agent until the pharmacologic agent is removed.

The hollow reservoir can then be filled with saline or another pharmacologic agent or drug.

### Intra Sclera Implantation of an Implantable Dispensing Device

As discussed above, published United States Patent Application US 2003/0064088 disclosed and described a drug-dispensing device which is hermetically sealed by buckling sutures or tissue adhesive glue to the target tissue to minimize leakage of a pharmacological agent during dispensing. Use of a tissue adhesive to affix the drug-dispensing device to the tissue being treated may result in blocking or occluding a dispensing port of the device. When such a drug dispensing device is applied over the sclera to deliver pharmacological agents to the retina or cholorid, the pharmacological agents must disuse through the entire thickness of the sclera before penetrating into the globe of the eye.

The implantable dispensing device as disclosed in the present invention can be configured to have the dispensing outlet located at the distal end of the dispensing device inserted into and tightly fitting within a sclera tunnel formed in the sclera. By doing so, the dispensing outlet is located within and sealed within the sclera tissue which reduces the thickness of the sclera tissue for which the pharmacological agent must diffused prior to entry into the portion of the eye to be treated.

The amount of a thickness of the sclera tissue through which the pharmacological agent may be varied by changing the depth at which the sclera tunnel is formed.

By inserting the implantable dispensing device intra scleral, the device can be configured to fit tightly into the tunnel defined by the scleral tissue preventing reflux of a pharmacological agent into the scleral tunnel. Thus, the scleral tissue eliminates the need for tissue adhesives or buckling sutures to seal the dispensing outlet or the device within the sclera.

The intra scleral placement of the implantable dispensing device reduces the barrier of penetration to the target tissue, e.g. macula, while inhibiting by reducing unwanted diffusion. This results in better efficacy and with fewer side effects or toxicities to surrounding or adjacent tissue. As such, the implantable device of the present invention may be used with pharmacological agent having a toxicity profile which could otherwise not be used or be administered in an episclera fashion.

An implantable dispensing device configured to be inserted into a tunnel formed in the outer tissue of an organ has advantage in that the formation of the tunnel and insertion of the device can be performed quickly resulting in a sealable, reliable tight fit of the implantable dispensing device into the tissue of the organ thereby enabling the dispensing outlet, which may be open, which may have a semi-permeable membrane or a permeable membrane, to effectively dispense a pharmacological agent directly into the tissue contiguous the dispensing outlet thereby increasing the efficacy of administration of the pharmacological agent, reducing the thickness of tissue to which the pharmacological agent must penetrate to reach the inter portion of the body to be treated and significantly reducing reflux of the administered pharmacological agent.

In FIG. 23, the pictorial representation of the layers of the eye between the sclera 320 and vitreous cavity 330 include the sclera 320, the choroid 324, the RPE complex 326, the retina 328 followed by the vitreous cavity 330. A tunnel showed by dashed lines 322 is formed within the sclera 320. In the alternative, a tunnel shown by dashed lines 322 could be an incision forming a flap which could be lifted to expose the interior of the sclera.

The pictorial representation of FIG. 24 shows a tunnel formed by dashed lines 322 which is formed to accommodate an implantable dispensing device 340 having a delivery tube 342 terminating in a deflected dispensing outlet 344. The deflected dispensing outlet 344 is inserted into and essentially implanted within the sclera 320.

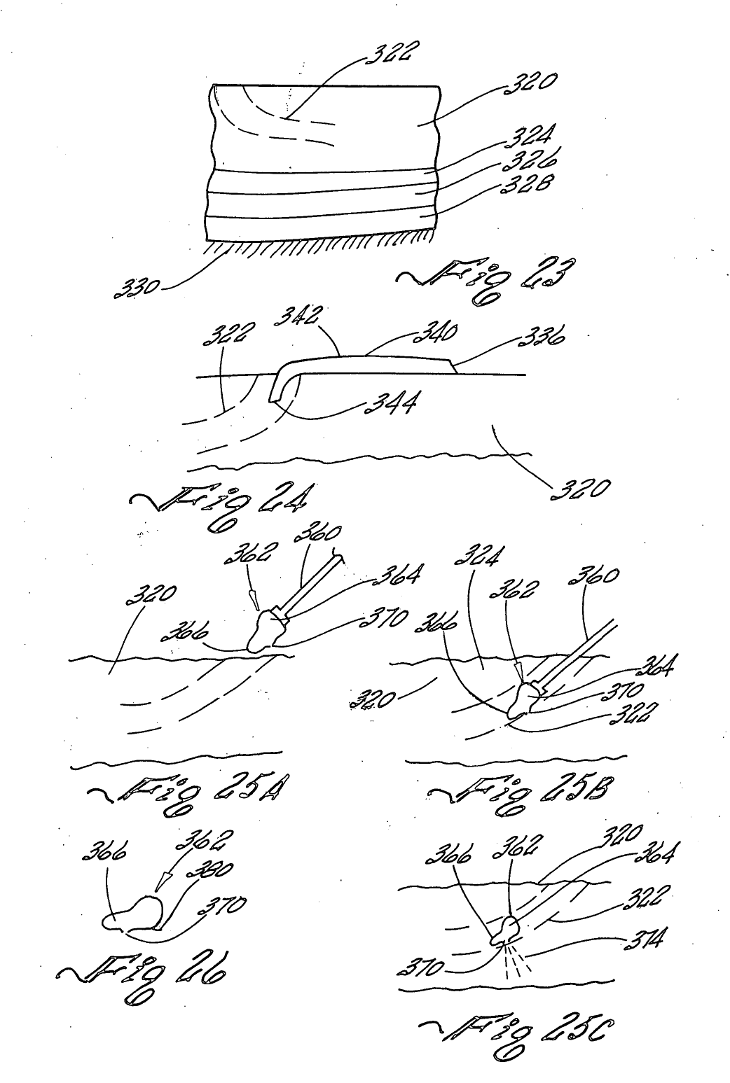

FIGS. 25A, 25B and 25C are pictorial representations of the sclera 320 having a tunnel formed therein as shown by dashed lines 322 to accommodate the implantation of a single use dispensing device shown by arrow 362 within the sclera 320. In FIG. 25A, an insertion tool 360 is shown having an implantable dispensing device 362 containing a pharmacological agent contained within the housing. In the embodiment of the dispensing device 362 as shown in FIGS. 25A, 25B and 25C, the dispensing device includes a hollow reservoir 364, a dispensing tube 366 terminating in a dispensing outlet 370. The embodiment of the dispensing device 362 is a single use device which has been filled with a slow release pharmacological agent which is configured to dispense the pharmacological agent into a sclera 320.

In FIG. 25A the insertion tool 360 having the dispensing device 362 is illustrated in a position above the tunnel showed by dashed lines 322 formed in the sclera 320 and in an insertion position.

FIG. 25B the insertion tool 360 having the implantable device 362 affixed thereto has been positioned at the desired location to implant the implantable dispensing device 362 within the tunnel shown by dashed line 322 within the sclera 320.

In FIG. 25C, the implantable dispensing device 362 has been implanted at the desired location in the sclera 320 within the tunnel shown by dashed lines 322 wherein the sclera 320 tissue surrounds and seals the dispensing outlet 370 of the dispensing device 362 within the tunnel shown by dashed lines 322 and the insertion tool 360 has been removed. Once the implantable device 362 is sealed in position by the sclera 320 the pharmacological agent is administered through the tissue of the sclera 324 as shown by dashed lines 374 which significantly increases the efficacy of the administration of the pharmacological agent to the sclera. The sealing action of the tissue of the sclera substantially prevents leakage of the pharmacological agent to surrounding tissue. The sclera 320 delivers the pharmacological agent to the inner portion of the eye to be treated which is below the sclera 320.

FIG. 26 is a pictorial representation of an embodiment of the insertable implantable dispensing device 362 which is filled with a slow release pharmacologic agent which could be in the form of a solid which slowly dissolves or breaks down into a particle size to be administered through the delivery tube 366 and administered through the dispensing outlet 370 into the tissue of the sclera 320 shown in FIG. 23.

The implantable dispensing device shown in FIG. 26 may include arcuate shaped extending members in the form of hooks shown as 380 which engage the walls of the tunnel formed in the sclera 320 to prevent retrograde of the device 362 from the sclera 320

Although the preferred embodiment of the implantable dispensing device is for treating an eye, several of the organs within the body such as for example the kidney, have a thick tissue exterior onto which or into which the implantable device can be affixed or implanted, as the case may be. For example, the implantable delivery device could be used to be implanted into an area of the body having cancer and a pharmacological agent configured for treating cancer could be administered directly to the desired portion of the body to enable the implantable delivery device to deliver a pharmacological agent or drug to a designated portion of the body. By implanting either the device entirely or the dispensing outlet in particular then the tissue, the tissue itself surrounds the implant or the outlet, as the case may be, sealing the same so that the pharmacological agent can be administered directly into the tissue thereby avoiding leakage to surround tissue.

It will be appreciated that various alterations and modifications may be made to the implantable refillable device or product to enhance the functional characteristics thereof. All such variations and modifications should be considered to fall within the scope of the invention as broadly hereinbefore described and as claimed hereafter.

All such uses, variations, modifications and the like are anticipated to be within the scope of this invention.

What is claimed is:

1. An implantable refillable device to deliver pharmacologic agents through a sclera to an internal portion of an eye comprising
a hollow reservoir;
a delivery tube having a first end and a second end, said first end communicating with the hollow reservoir and said second end terminating in a dispensing outlet and wherein said delivery tube is configured in a selected shape to extend from the hollow reservoir anterior in an eye to a posterior segment in an eye adjoining a sclera;
said second end being configured to position said dispensing outlet in contact with or contiguous a sclera and being located posteriorly in an eye and proximate to an internal portion of an eye to be treated with a pharmacologic agent.

2. The implantable refillable device of claim 1 wherein said dispensing outlet is in the form of an unobstructed opening to enable a pharmacologic agent to pass therethrough.

3. The implantable refillable device of claim 1 further comprising
a permeable membrane located in said dispensing outlet and wherein said permeable membrane is selected to have a porosity to pass a pharmacologic agent therethrough and a structural integrity to resist tissue from the outer surface portion of the sclera migrating in to and occluding the dispensing outlet.

4. The implantable refillable device of claim 1 wherein said delivery tube has a selected distance between said first end and said second end to position said dispensing outlet on the sclera and curved inferior of the inferior oblique muscle to position said dispensing outlet to overlie the macula region of the sclera to enable a pharmacologic agent to pass through the sclera and into the macula.

5. The implantable refillable device of claim 1 wherein said delivery tube has a selected distance between said first end and said second end to position said dispensing outlet having a selected surface area in contact with or contiguous sclera on a posterior segment of the eye, beneath two rectus muscles and positioned to overlie the macula region of the sclera enabling the dispensing outlet to administer a pharmacologic agent over a large surface area of the sclera overlaying the macula region of the sclera to enable a pharmacologic agent to pass through the sclera and into the macula.

6. The implantable refillable device of claim 1 wherein said delivery tube has a selected distance between said first end and said second end to position said dispensing outlet on the sclera adjacent to an optic nerve of an eye to enable a pharmacologic agent to pass through the sclera and into the optic nerve.

7. The implantable refillable device of claim 1 wherein said delivery tube has a selected distance between said first end and said second end to position said dispensing outlet on the sclera located proximate the optic nerve of an eye to enable a pharmacologic agent to pass through the sclera into the optic nerve.

8. The implantable refillable device of claim 1 wherein said said dispensing outlet is configured to be placed circumferentially around a portion of the sclera of an optic nerve of an eye to enable a pharmacologic agent to pass through the sclera into the optic nerve.

9. The implantable refillable device of claim 1 wherein said delivery tube has a selected distance between said first end and said second end to position said dispensing outlet on the sclera proximate the optic nerve of an eye to enable a pharmacologic agent to pass therethrough and into the sclera surrounding the optic nerve to enable a pharmacologic agent to pass through the sclera and into the optic nerve.

10. The implantable refillable device of claim 9 wherein said dispensing outlet is configured to be placed circumferentially around a portion of the sclera surrounding an optic nerve of an eye to enable a pharmacologic agent to pass through the sclera into the optic nerve.

11. The implantable refillable device of claim 1 wherein said hollow reservoir has an internal cavity and a self-sealing needle insertion section to enable a needle to be passed through said needle insertion section and into the internal cavity to enable filling of the internal cavity with a pharmacologic agent to be dispensed in an eye.

12. The implantable refillable device of claim 1 wherein the implantable refillable device is configured to have one or more needles inserted sequentially into and removed from the self-sealing needle insertion section for administrating repeated juxtasclera injections into the posterior segment of the eye to treat an internal portion of an eye.

13. The implantable refillable device of claim 1 wherein the implantable refillable device is treated or impregnated with pharmacologic agents to minimize fibrosis and scaring around the device.

14. The implantable refillable device of claim 1 wherein said hollow reservoir has an internal cavity and a self-sealing needle insertion section to enable a needle to be passed through said needle insertion section and into the internal cavity containing a pharmacologic agent to be dispensed in an eye to enable withdrawing of a pharmacologic agent from the internal cavity.

15. The implantable refillable device of claim 1 wherein said hollow reservoir has an internal cavity and a self-sealing needle insertion section, said needle insertion section being configured to retain a pharmacologic agent located with the internal cavity to prevent refluxing of a pharmacologic agent over the eye and being positioned relative to the anterior portion of an eye to enable a needle to be passed through said needle insertion section and into the internal cavity to enable filling of the internal cavity with a pharmacologic agent to be used for treating an eye.

16. The implantable refillable device of claim 1 wherein said hollow reservoir has an internal cavity, a needle insertion section and an impermeable shield spaced distally from the needle insertion section to prevent further penetration of a needle of a syringe after entering the needle insertion section to inhibit a syringe needle from passing therethrough and into an eye.

17. The implantable refillable device of claim 1 wherein said hollow reservoir has an internal cavity, a needle insertion section and an impermeable shield spaced distally from the needle insertion section, said needle insertion section being configured to retain pharmacologic agent located with the internal cavity from refluxing over the eye and being positioned relative to the anterior portion of an eye to enable a syringe needle to be passed through said needle insertion section and into the internal cavity to enable filling of the internal cavity and wherein said impermeable shield prevents a syringe needle from passing therethrough into an eye.

18. The implantable refillable implant of claim 1 wherein eyelet for suturing the device to a sclera are formed in an anterior section of the device.

19. The implantable refillable implant of claim 1 wherein a medical adhesive is applied to that portion of the device to be placed in contact with or contiguous a sclera.

20. An implantable refillable device to deliver drugs through a posterior outer surface of an eye to an internal portion of an eye comprising
a hollow reservoir, said hollow reservoir having an internal cavity and a needle insertion section, said needle insertion section being configured to retain a drug located within the internal cavity from refluxing over the eye and being positioned relative to the anterior portion of an eye to enable a syringe needle to be passed through said needle insertion section and into the internal cavity to enable filling of the internal cavity with a drug and withdrawing of a drug from the internal cavity; and
a delivery tube having a proximal end and a distal end, said proximal end communicating with the hollow reservoir and said distal end terminating in a drug dispensing outlet wherein said delivery tube is configured in a generally curved shape to extend from the hollow reservoir anterior in an eye to a posterior segment posterior in an eye adjoining a sclera;
said second end being configured to position said drug dispensing outlet in contact with or contiguous a posterior outer surface of an eye.

21. The implantable refillable device of claim 20 wherein said hollow reservoir further comprises
an impermeable shield spaced distally from the needle insertion section to prevent further penetration of a needle of a syringe after entering the needle insertion section to inhibit a syringe needle from passing therethrough and into an eye.

22. The implantable refillable device of claim 20 wherein said dispensing outlet is positioned in contact with or contiguous the sclera of an eye above the macula.

23. The implantable refillable device of claim 20 wherein said dispensing outlet is positioned in contact with or contiguous the sclera of an eye above the optic nerve.

25. The implantable refillable device of claim 20 wherein said dispensing outlet is configured to be placed circumferentially around a portion of the outer surface of an optic nerve of an eye.

26. An implantable refillable device to deliver a pharmacologic agent directly through an outer surface of an eye to an internal portion of an eye to be treated comprising
a hollow reservoir;
a delivery tube having a first end and a second end, said first end communicating with the hollow reservoir and said second end terminating in a pharmacologic agent dispensing outlet wherein said delivery tube is configured in a shape to extend from the hollow reservoir anterior in an eye to a posterior segment posterior in an eye and adjoining a sclera;
said second end being configured to be position said a pharmacologic agent-dispensing outlet in contact with or contiguous a sclera of an eye locate above an internal portion of an eye to be treated with a pharmacologic agent.

27. An implantable refillable device of claim 26 wherein said dispensing outlet is positioned in contact with or contiguous a sclera of an eye proximate the macula.

28. An implantable refillable device of claim 26 wherein said dispensing outlet is positioned in contact with or contiguous a sclera of an eye proximate the optic nerve.

29. An implantable refillable device of claim 26 wherein said dispensing outlet is positioned in contact with or contiguous a sclera surrounding the optic nerve.

30. A method for treating an eye comprising the steps of
forming an implantable refillable device to deliver a pharmacologic agent to an internal portion of an eye having a hollow reservoir and a delivery tube having a first end and a second end wherein said first end communicates with the hollow reservoir, wherein said delivery tube is configured in a shape to extend from the hollow reservoir anterior in an eye to a posterior segment posterior in an eye adjoining a sclera and wherein said second end terminates in a dispensing outlet to be position in contact with or contiguous a sclera of an eye; and
surgically implanting said device under the Tenon's capsule and in contact with or contiguous the sclera of an eye with the hollow reservoir positioned anteriorly on the sclera at a location where a needle can be inserted into the hollow reservoir and with said dispensing outlet being is located posteriorly on the sclera and proximate the location of an internal portion of an eye to be treated by a pharmacological agent.

31. The method for treating an eye of claim 30 wherein said step of surgically implanting includes positioning said dispensing outlet in contact with or contiguous the sclera proximate the macula of an eye.

32. The method for treating an eye of claim 30 wherein said step of surgically implanting includes positioning said dispensing outlet in contact with or contiguous the sclera proximate the optic nerve of an eye.

33. The method for treating an eye of claim 30 wherein said step of surgically implanting includes positioning said dispensing outlet in contact with or contiguous a sclera proximate the optic nerve of an eye.

34. The method for treating an eye of claim 30 wherein said step of surgically implanting includes positioning said dispensing outlet in contact with or contiguous a sclera surrounding the optic nerve of an eye.

35. The method for treating an eye of claim 30 further comprising the step of
injecting through a needle into the hollow reservoir a pharmacologic agent to be used for treating an eye.

36. The method for treating an eye of claim 30 further comprising the step of
injecting through a needle into the hollow reservoir a controlled released drug to be used for treating an eye.

37. The method for treating an eye of claim 36 further comprising the step of
injecting through a needle into the hollow reservoir a controlled released drug configured in the form of micro-spheres having a known dissolution rate for providing a controlled rate of drug delivery through the dispensing outlet for treating an eye.

38. The method for treating an eye of claim 30 further comprising the step of
injecting through a needle into the hollow reservoir a pharmacologic agent comprising anacortate acetate which penetrates through the sclera.

39. The method for treating an eye of claim 36 further comprising the step of
withdrawing through a needle from the hollow reservoir a pharmacologic agent by the steps of alternating injecting sterile air through the needle injection section and withdrawing the pharmacologic agent through the needle.

40. A method for removing a pharmacologic agent from an implant having a needle insert section and a hollow reservoir comprising the steps of:
inserting a needle through the needle insertion section;
injecting a small amount of sterile air through the needle into the hollow reservoir;
allowing bubbles of sterile air to rise to the superior part of the hollow reservoir;
withdrawing an appropriate quantity of pharmacologic agent from the hollow cavity using the needle; and
alternating injecting another small amount of sterile air through the needle into the hollow reservoir and withdrawing through the needle an appropriate quantity of pharmacologic agent until the pharmacologic agent is removed.

41. The method of claim 40 further comprising the step of filling the hollow reservoir with saline or another pharmacologic agent or drug.

42. The method of claim 40 wherein the implant is located on the sclera of an eye and further comprising the step of:
placing the eye in a dependent location.

43. An implantable device to deliver pharmacologic agents through an outer surface tissue of an organ comprising a hollow reservoir;
a delivery tube having a dispensing outlet and wherein said delivery tube is configured in a selected shape to extend from the hollow reservoir to a selected portion of the outer surface tissue of an organ;
said dispensing outlet being configured to be positioned in contact with or contiguous an outer tissue of an organ and proximate to an internal portion of a body to be treated with a pharmacologic agent.

44. An implantable delivery device for delivering a therapeutic agent into a target tissue comprising
an enclosed therapeutic agent container having a dispensing outlet, said container being configured to be implanted in a tunnel or flap of the outer tissue of an organ wherein the dispensing outlet is engaged by and sealed within the tunnel by the outer tissue of an organ enabling administration of a therapeutic agent into the target tissue contiguous to the dispensing outlet.

45. A method for treating an inner portion of an organ within a body having an outer tissue enclosing the organ comprising the steps of:
forming a tunnel or flap in the outer tissue surrounding the organ; and
implanting within said tunnel or flap an implantable delivery device for delivering a therapeutic agent into a target tissue wherein said implantable delivery device includes a therapeutic agent container having a dispensing outlet and wherein said container is configured to be implanted into a tunnel or flap of the outer tissue of an organ and wherein the dispensing outlet is engaged by and sealed within the tunnel by the outer tissue of an organ enabling administration of a therapeutic agent into the target tissue contiguous to the dispensing outlet.

46. The method of claim 45 further comprising a step of:
removablely placing the implantable dispensing device onto the end of an insertion tool.

47. The method of claim 46 further comprising a step of:
inserting the implantable dispensing device located at the end of an insertion tool into the tunnel or flap of the outer tissue and advancing the implantable dispensing device to a desired location within the tunnel or flap of the outer tissue.

48. The method of claim 47 further comprising a step of:
separating the implantable dispensing device from the insertion tool and withdrawing the insertion tool from the tunnel or flap of the outer tissue.
